(19) United States
(12) Patent Application Publication
(10) Pub. No.: US 2003/0130348 A1
(43) Pub. Date: Jul. 10, 2003

(54) Polyunsaturated oil saponification

(76) Inventors: George K. Strohmaier, Medina, OH (US); Eiler D. Frederiksen, Henderson, NV (US)

Correspondence Address: Synnestvedt & Lechner, LLP, Suite 2600, 1101 Market Street, Philadelphia, PA, 19107-2950, US

(21) Appl. No.: 10/299,337

(22) Filed: Nov. 18, 2002

Related U.S. Application Data
(60) Provisional application No. 60/334,471, filed on Nov. 16, 2001.

(51) Int. Cl. A61K 31/202; C07C 53/126

(52) U.S. Cl. 514/560; 554/224

(57) ABSTRACT

Free-flowing fatty acid calcium salt products are discclosed containing at least one omega-3 fatty acid selected from DHA, EPA, DPA and LA, wherein each fatty acid, when present, is at a level between about 1 and about 25% by weight. The preparation of fatty acid calcium salts from high glyceride content polyunsaturated oils by saponification processes is also disclosed.

### CROSS-REFERENCE TO RELATED APPLICATION

The present application claims priority benefit of U.S. Provisional Patent Application Serial No. 60/334,471 filed Nov. 16, 2001, the disclosure of which is incorporated by reference.

### BACKGROUND

The present invention relates to the preparation of fatty acid calcium salts from high glyceride content polyunsaturated oils such as fish oils. The fatty acid calcium salts are rich in desirable omega-3 fatty acids, including eicosapentanoic acid (EPA), decosahexanoic acid (DHA), decosapentanoic acid (DPA) and linolenic acid (LA).

Omega-3 fatty acids are known to promote animal fertility, and have other nutritional and physiological properties as well. The present invention therefore also relates to fatty acid calcium salts prepared by the method of the present invention high in desirable omega-3 fatty acids, and the use of these calcium salts as animal nutritional supplements. The fatty acid calcium salts can be used to enrich the omega-3 fatty acid content of a variety of feed or food products, including pet food products.

The enrichment of food products with omega-3 fatty acids has proven to be problematic. The addition of polyunsaturated oils to certain feed or food products to enhance their omega-3 fatty acid content produces an unacceptable yield loss in the levels of the omega-3 fatty acids desired. This is proven to particularly be a problem with enriching the omega-3 fatty acid content of pet foods.

Fatty acid calcium salts are known rumen-inert feed supplement for ruminants such as cattle. Fatty acids in calcium salt form are protected from microbial action in the rumen. Fatty acid calcium salt rumen bypass feed supplements are disclosed by U.S. Pat. Nos. 4,642,317; 4,826,694; 4,853,233 and 4,909,138. The disclosures of all four patents are incorporated herein by reference.

### DESCRIPTION OF THE INVENTION

It has now been discovered that the omega-3 fatty acid yield loss is solved by converting the pure polyunsaturated oils to fatty acid calcium salts and using these calcium salts instead as the nutritional supplements. However, it is not commercially feasible to use the processes disclosed by the above-listed patents to prepare fatty acid calcium salts from polyunsaturated oils because of their high glyceride content.

Polyunsaturated oils such as fish oils have glyceride contents as high as 100%. That is, all of the fatty acids can be in the glyceride form. Fatty acid glycerides do not readily react to form calcium salts using the processes disclosed by the above-listed patents. For a product to be commercially feasible, glyceride levels below about 5 weight percent are desirable to produce a free-flowing and stable product. The processes disclosed by the above-listed patents produce fatty acid calcium salts having much higher levels of unreacted glyceride. Products with residual glyceride contents above 5 weight percent lack storage stability, and are susceptible to oxidation, post-heating, melting, subsequent product solidification, and a tendency to form lumps upon storage.

U.S. patent application Ser. No. 09/990,784 discloses the preparation of fatty acid calcium salts from fatty acid feedstocks having glyceride contents as high as 60% by weight. The disclosure of this application is incorporated herein by reference. The process disclosed by this patent application can be used to prepare fatty acid calcium salts from polyunsaturated oils in general by diluting the glyceride content to about 60 weight percent or lower with a low glyceride content feedstock such as Palm Fatty Acid Distillate (PFAD). While the process disclosed by that patent application can be used to prepare fatty acid calcium salts rich in omega-3 fatty acids, there remains a need for fatty acid calcium salts having higher levels of omega-3 fatty acids.

U.S. Pat. No. 6,229,031 discloses a high temperature saponification process for the preparation of fatty acid calcium salts from feedstocks containing glyceride contents as high as 100 weight percent. It has now been determined that this process can be used to prepare fatty acid calcium salts from polyunsaturated oils when omega-3 fatty acid levels are needed higher than that which can be prepared by the process disclosed by U.S. patent application Ser. No. 09/990,784. In particular, the high temperature process of U.S. Pat. No. 6,229,031 can be used to prepare fatty acid calcium stocks from feedstocks having glyceride contents greater than about 60 weight percent up to 100% glyceride content oils. Therefore, for purposes of the present invention polyunsaturated oils are defined as having a glyceride content between about 60 and about 100 weight percent, preferably between about 70 and about 100 weight percent, more preferably between about 80 and about 100 weight percent, and even more preferably between about 90 and about 100 weight percent, with a glyceride content of 100% being most preferred.

The purpose of the present invention is to provide fatty acid calcium salts of polyunsaturated fatty acids from feedstocks for those acids that are typically very high in glyceride content, without have to significantly dilute the feedstock, if at all, with low glyceride content feedstocks that contain little, if any, polyunsaturated fatty acids. Thus essentially any polyunsaturated oil containing useful levels of polyunsaturated fatty acids is suitable for use with the present invention, and for purposes of the present invention is included within the definition of a polyunsaturated oil. Stated another way, if an oil with a glyceride content between 60 and 100 weight percent has utility as a polyunsaturated oil, it is within the definition of high glyceride content polyunsaturated oils for purposes of the present invention.

The present invention is advantageously used with polyunsaturated oils having a low level of polyunsaturated fatty acids because with the present invention the polyunsaturated fatty acid concentration is essentially conserved. Calcium salts of polyunsaturated oils containing between about 3 and about 100 weight percent unsaturated fatty acids based on the total fatty acid content can be formed using the methods of the present invention. Oils with lower levels of polyunsaturated fatty acids may be used if they have utility based on their polyunsaturated fatty acid content.

Thus, the process of U.S. Pat. No. 6,229,031 can be used to prepare fatty acid calcium salts from pure polyunsaturated oils such as fish oils, and from polyunsaturated oils that have been diluted with lower glyceride content feedstocks such as PFAD down to glyceride contents as low as about 60 weight percent. The disclosure of U.S. Pat. No. 6,229,031 is also incorporated herein by reference. Essentially any polyunsaturated oil of animal, vegetable or fish origin, including those disclosed in the above-referenced U.S. patent application Ser. No. 09/990,784 can be used with the present invention, either in pure form or diluted to lower the glyceride content. Examples of suitable oils include soybean oil, cottonseed oil, flax seed oil, rape seed oil, and the like.

Accordingly, the process of U.S. Pat. No. 6,299,031 can be used to prepare fatty acid calcium salts from fish oil sources such as menhaden, herring, mackerel, caplin, tilapia, tuna, sardine, pacific saury, krill, and the like, as well as from polyunsaturated oils other that fish oils. The fatty acid calcium salts include calcium salts containing from about 1 to about 20% by weight of DHA, and/or from about 1 to about 20% by weight of EPA, and/or from about 1 to about 20% by weight of DPA, and/or from about 1 to about 20% by weight of LA. Other conventional biologically active ingredients can be added to the calcium salt products at conventional levels by known means.

The polyunsaturated oils can be unstable if over-processed under the saponification conditions disclosed by U.S. pat. No. 6,299,031. Preferred methods according to the present invention hold the polyunsaturated oil at ambient or below ambient temperature conditions, more preferably between about 10 and about 30 degrees C. Preferred methods also additionally blanketing the headspace with mixtures of inert gases to eliminate oxygen. It is also preferred to blend an effective amount of one or more antioxidants and chelating agents for polyunsaturated oils into the admixture to be reacted.

It is also preferable to preheat the admixture in an appropriate heat exchange device immediately prior to the high temperature reactor, and also to blend the admixture with metallic rare earth metal oxides or their hydrated forms. Stability of the calcium salts can also be improved by limiting the reaction time and temperature. The preferred reaction temperature is between about 150 and about 250 degrees C., and the preferred reation time within this temperature range is between about 2 and about 30 minutes.

A preferred step for maintaining product stability is to run the reaction at either reduced pressures, vacuum conditions, or in the presence of an inert gas mixture such as N₂ or CO₂ or other. This will prevent the oxidation at the elevated temperatures that will destabilize the polyunsaturated fatty acids in calcium salt form. Another preferred step for maintaining stability is to immediately cool the product as quickly as possibly while simultaneously flaking the material into a free flowing granular product with minimal fines.

While the calcium salts of the present invention can be used as rumen-inert feed supplements for ruminants such as cattle, the calcium salts are also useful in general as nutritional supplement for humans, other mammals, and non-mammals, including birds and fish.

Thus, methods in accordance with the present invention add an effective amount of the fatty acid calcium salts of the present invention to a food product including pet food products such as cat food and dog food. Effective amounts include amounts that will provide a food product having an omega-3 fatty acid content between about 0.05 and about 1.5 weight percent. Omega-3 fatty acid content between about 0.1 weight percent and about 0.5 weight percent are preferred. The present invention also includes food products containing the fatty acid calcium salts of the present invention within the ranges described, including pet food products such as cat food and dog food. The cat foods and dog foods include dry, semi-moist and moist cat food and dog food prepared by otherwise conventional methods from conventional formulations incorporating conventional pet food ingredients.

The fatty acid calcium salts of the present invention can be used in the fertility enhancement methods disclosed by WO 99/66877, which is incorporated herein by reference. The present invention therefore includes methods according to that publication using the calcium salts of the present invention.

The present invention thus provides a means by which omega-3 fatty acid rich calcium salts may be prepared having utility as nutritional supplements for essentially any animal for which omega-3 fatty acids provide nutritional or therapeutic benefit.

What is claimed is:

1. A free-flowing fatty acid calcium salt product comprising at least one omega-3 fatty acid selected from the group consisting of DHA, EPA, DPA and LA, wherein each fatty acid, when present, is at a level between about 1 and about 25% by weight
